(19) United States
(12) Patent Application Publication
(10) Pub. No.: US 2025/0154454 A1
(43) Pub. Date: May 15, 2025

(54) MICROFLUIDIC SYSTEMS AND METHODS FOR ISOLATING TARGET ENTITIES

(71) Applicant: The General Hospital Corporation, Boston, MA (US)

(72) Inventors: Mehmet Toner, Charlestown, MA (US); Gloria Lee, Norwood, MA (US); Baris Ragip Mutlu, Cambridge, MA (US)

(21) Appl. No.: 18/722,278

(22) PCT Filed: Dec. 20, 2022
(86) PCT No.: PCT/US2022/053505

§ 371 Date: Jun. 20, 2024

Related U.S. Application Data
(60) Provisional application No. 63/291,928, filed on Dec. 20, 2021.

(51) Int. Cl. C12N 5/075; B01L 3/00

(52) U.S. Cl. CPC C12N 5/0609; B01L 3/502761; B01L 2200/0652; B01L 2400/086

(57) ABSTRACT

The present disclosure features systems and methods for isolating target entities (TEs) found in target entity-secondary entity complexes within a biological fluid by exploiting interactions between TE, secondary entities (SEs), and/or engineered surfaces (ESs). TE isolation involves a two-step process in which TE-SE complexes are initially captured using, e.g., size-based approaches or binding interactions between SEs and ESs, and then TEs are specifically released from the captured TE-SE complexes using, e.g., biochemical means, such as disassociating enzymes and/or binding inhibitors, or using physical properties of fluid flow, e.g., flow velocity and/or shear rate.

### TECHNICAL FIELD

This disclosure relates to the isolation of rare entities and/or rare entity clusters, for example, cumulus oocyte complexes (COC).

### BACKGROUND

Target entities (TEs), such as cells, are often isolated from biological fluids by exploiting interactions between surface markers (e.g., proteins and antigens) of the TEs and engineered (e.g., antibody-coated) surfaces of fluidic devices or through use of magnetic beads. In some biological fluids, TEs can interact with secondary entities (SEs), e.g., secondary cells, and form complexes (TE-SE complexes) based on such interactions.

### SUMMARY

Systems and techniques are described herein for isolating TEs found in TE-SE complexes within a biological fluid by exploiting interactions between TEs, SEs, and engineered surfaces (ESs). The capture and release mechanisms of many known TE isolation processes involve using interactions (e.g., antigen-antibody interactions) between the TEs and SEs, thereby resulting in an entity-specific isolation paradigm. In contrast, the techniques disclosed herein enable targeting of different interactions in each stage (capture, release) while still enabling entity-specific isolation of TEs. This approach provides several advantages, such as allowing non-specific interactions to be used in either capture or release stages since the combination yields an overall entity-specific isolation paradigm. For example, non-specific interactions between SEs and engineered surfaces can be used for high efficiency capture of TE-SE complexes, and a specific interaction between TEs and SEs can be used for releasing TEs, while still enabling entity-specific TE isolation.

Additionally, the techniques disclosed herein can exploit a number of available interactions to increase TE capture and release. For example, in some isolation processes, the available interactions are often limited to TEs and SEs, while the techniques disclosed herein can enable a three-fold increase: (i) TE-engineered surface interactions, (ii) SE-engineered surface interactions, and (iii) TE-SE interactions. For example, SE-engineered surface interactions can be used for capturing TE-SE complexes and TE-SE interactions can be used for releasing the TEs.

Size-based capture methods (e.g., using microfilters) can also be used to capture TE-SE complexes and other non-targeted biological debris in the same size range. Specific interactions between TEs and SEs can then be targeted to weaken or break bonds between TEs and SEs. An increased flowrate can then be used to enable entity-specific release of TEs given the weakened interactions within the TE-SE complex. Some examples of the disclosed techniques provide the capture and isolation of rare entities, e.g., cells, and/or rare entity, e.g., cell, clusters from a fluid sample. For example, the disclosed techniques can enable the isolation of oocytes or COCs from follicular fluid (FF) for use in in vitro fertilization (IVF) or assisted reproductive technologies (ART). As detailed herein, the techniques can involve various processes that combine specific or non-specific capture/release of TE (e.g., oocytes, COC), secondary entities (e.g., cumulus cells (CCs), granulosa cells (CGs), and engineered surfaces (ESs).

Additionally, the techniques disclosed herein can exploit the number of available interactions to increase TE capture and release. For example, in some isolation processes, the available interactions are often limited to TEs and SEs, while the techniques disclosed herein can enable a three-fold increase: (i) TE-engineered surface interactions, (ii) SE-engineered surface interactions, and (iii) TE-SE interactions. For example, SE-engineered surface interactions can be used for capturing TE-SE complexes and TE-SE interactions can be used for releasing the TEs.

In one general aspect, the disclosure provides methods of extracting target entities from a sample fluid including target entities bound to one or more secondary entities by a specific binding interaction to form target entity-secondary entity complexes that include at least one target entity. The method includes capturing one or more target entity-secondary entity complexes, if any, in the sample fluid. The capturing is accomplished using a size-based capture mechanism in a microfluidic device. The microfluidic device includes a channel having an inlet and an outlet and two or more arrays of structures arranged within the channel between the inlet and the outlet. A first array is arranged in the channel closer to the inlet than a second array. Structures in the first array are arranged further apart than structures in the second array. Structures in the second array are arranged at a distance apart that enables capture of target entity-secondary entity complexes, but provides sufficient space to allow target entities and secondary entities to flow through and out of the size-based capture mechanism. The capturing is also accomplished using a binding interaction between the secondary entities and a binding agent in a microfluidic device including a channel having an internal surface, an inlet, and an outlet. The binding agent is attached to the internal surface and binds specifically or non-specifically to the secondary entities. The capturing may also be accomplished using both techniques.

The method includes flowing a reagent through the microfluidic device to weaken or break the specific binding interaction between the target entities and the secondary entities or weaken or break binding interactions within or between the secondary entities, thereby releasing the target entities from the target entity-secondary entity complexes. The method also includes capturing a portion of the reagent that includes the target entities.

One or more implementations can include the following optional features. For example, in some implementations, the sample fluid includes a biological fluid. The target entities include target cells. The secondary entities specifically bind to the target cells. The reagent includes a substance that inhibits the specific binding between the target cells and the secondary entities or inhibits binding interactions within or between the secondary entities.

In some implementations, the sample fluid includes follicular fluid. The target entities include oocytes or cumulus oocyte complexes (COC). The secondary entities include cumulus cells or granulosa cells. The reagent includes hyaluronidase.

In some implementations, the sample fluid includes a dissected ovarian tissue sample collected from a mammal. The mammal includes a human, a sheep, a horse, or a bovine.

In some implementations, the binding agent non-specifically binds the secondary entities to the internal surface. The reagent specifically weakens specific interactions of the target entities with the secondary entities, thereby releasing the target entities from the target entity-secondary entity complexes.

In some implementations, the binding agent includes poly-L-lysine or laminin and the reagent includes hyaluronidase.

In some implementations, the binding agent specifically binds the secondary entities to the internal surface. The reagent specifically weakens specific interactions of the target entities with the secondary entities, thereby releasing the target entities from the complexes.

In some implementations, the binding agent includes follicle-stimulating hormone receptor (FSHR) antibody, luteinizing hormone choriogonadotropin receptor (LHCGR) antibody, or Anti-Mullerian hormone receptor type 2 (AMHR2) antibody. The reagent includes hyaluronidase.

In some implementations, the binding agent promotes specific binding of the secondary entities and the internal surface.

In some implementations, the binding agent includes follicle-stimulating hormone receptor (FSHR) antibody, luteinizing hormone choriogonadotropin receptor (LHCGR) antibody, or Anti-Mullerian hormone receptor type 2 (AMHR2) antibody. Flowing the reagent through the microfluidic device also includes flowing the reagent through the microfluidic device at a flow rate sufficient to (i) weaken or break the specific binding interaction between the target entities and the secondary entities and (ii) maintain binding of non-target entities bound to the internal surface.

In some implementations, the channel includes magnetic beads functionalized for binding to the target entities. In such implementations, the method further includes applying a magnetic field to the microfluidic device to sort magnetic beads bound to the target entities within the channel. Additionally, the portion of the reagent that includes the target entities is captured based on the sorting of magnetic beads bounds to the target entities within the channel.

In some implementations, the size-based capture mechanism further includes a third array and a fourth array. The third array is arranged between the first array and the second array. The fourth array is arranged between the third array and the second array. The structures included in the third array are separated by gap sizes that are (i) larger than gap sizes between the structures included in the second array and (ii) smaller than gap sizes between the structures included in the first array. The structures included in the fourth array are separated by gap sizes that are (i) larger than gap sizes between the structures included in the second array and (ii) smaller than gap sizes between the structures included in the third array.

In some implementations, the structures included in the first array have gap sizes between structures of 700-1200 μm. The structures included in the second array have gap sizes between structures of 75-150 μm. The structures included in the third array have gap sizes between structures of 350-650 μm. The structures included in the fourth array have gap sizes between structures of 150-300 μm.

In some implementations, a height of the structures included in the first array, the second array, the third array, and/or the fourth array is between 100-1000 μm. For example, the height is approximately 350 μm.

In another general aspect, the disclosure provides microfluidic devices for removing one or more non-target entities from a sample fluid including target entities bound to one or more secondary entities by a specific binding interaction to form target entity-secondary entity complexes that include at least one target entity. The microfluidic device includes a channel having an inlet, an outlet, an internal surface, and two or more arrays of structures arranged within the channel between the inlet and the outlet. A first array is arranged in the channel closer to the inlet than a second array. Structures in the first array are arranged further apart than structures in the second array. A binding agent is attached to the internal surface and/or to one or more of the structures within the first array or the second array or both the first and second arrays. The binding agent binds specifically or non-specifically to the secondary entities.

In some implementations, the microfluidic device further includes a third array of structures and a fourth array of structures. The third array is arranged in the channel between the first array and the second array. The fourth array is arranged in the channel between the third array and the second array. The structures included in the third array are separated by gap sizes that are (i) larger than gap sizes between the structures included in the second array and (ii) smaller than gap sizes between the structures included in the first array. The structures included in the fourth array are separated by gap sizes that are (i) larger than gap sizes between the structures included in the second array and (ii) smaller than gap sizes between the structures included in the third array.

In some implementations, the structures included in the first array have gap sizes between structures of 700-1200 μm. The structures included in the second array have gap sizes between structures of 75-150 μm. The structures included in the third array have gap sizes between structures of 350-650 μm. The structures included in the fourth array have gap sizes between structures of 150-300 μm.

In some implementations, a height of the structures included in the first array, the second array, the third array, and the fourth array is between 100-1000 μm. For example, the height is approximately 350 μm.

In some implementations, a second channel in fluid communication with the channel and having an inlet, a first outlet, a second outlet, a third outlet, and two rows of filtering structures arranged within the second channel between the inlet and the third outlet. A first row of filtering structures is spaced apart in the second channel from a second row of filtering structures such that spacing between the first row and the second row defines a central portion of the second channel. Structures in the first row and the second row are spaced apart along a direction of flow in a central portion so as to permit non-target entities to flow through spaces between the structures and out the first outlet or the second outlet of the second channel. The first row and the second row are spaced apart perpendicular to the direction of flow in the central portion. The central portion provides sufficient space to allow target entities and secondary entities to flow through and out of the third outlet of the second channel. A third channel in fluid communication with the second channel and having an inlet, a product outlet, and three or more arrays of structures arranged within the third channel between the inlet and the product outlet. Structures in the three or more arrays are arranged at a distance apart to release target entity-secondary entity complexes through and out of the product outlet of the third channel.

In some implementations, structures included in the first row and the second row are sized to have a length between 650-850 μm along the direction of flow in the central portion. Structures included in the first row and the second row are spaced apart between 30-60 μm along the direction of flow of in the central portion. The central portion has a length between 400-600 μm perpendicular to the direction of flow in the central portion.

In some implementations, a height of the structures included in the first row and the second row is between 100-1000 μm. For example, the height is approximately 350 μm.

In some implementations, the three or more arrays of structures arranged within the third channel includes five arrays of structures. A first array of the five arrays of structures is arranged in the third channel closer to the inlet than a second array of the five arrays of structures. A third array of the five arrays of structures is arranged in the third channel between the first array and the second array. A fourth array of the five arrays of structures is arranged in the third channel between the third array and the second array. A fifth array of the five arrays of structures is arranged in the third channel between the fourth array and the second array. Structures included in the first array are arranged further apart than structures in the second array. Structures included in the third array are separated by gap sizes that are (i) larger than gap sizes between the structures included in the second array, the fourth array, and the fifth array, and (ii) smaller than gap sizes between the structures included in the first array. Structures included in the fourth array are separated by gap sizes that are (i) larger than gap sizes between the structures included in the second array and the fifth array, and (ii) smaller than gap sizes between the structures included in the third array. Additionally, structures included in the fifth array are separated by gap sizes that are (i) larger than gap sizes between the structures included in the second array, and (ii) smaller than gap sizes between the structures included in the fourth array.

In some implementations, the structures included in the first array have gap sizes between structures of 95-145 μm. The structures included in the second array have gap sizes between structures of 5-55 μm. The structures included in the third array have gap sizes between structures of 75-125 μm. The structures included in the fourth array have gap sizes between structures of 55-105 μm. The structures included in the fifth array have gap sizes between structures of 25-75 μm.

In some implementations, a height of the structures included in the first array, the second array, the third array, the fourth array, and/or the fifth array is between 100-1000 μm. For example, the height is approximately 350 μm.

In another general aspect, the disclosure provides microfluidic devices for extracting target entities from a sample fluid including target entities bound to one or more secondary entities by a specific binding interaction to form target entity-secondary entity complexes that include at least one target entity. The microfluidic device includes a first channel having an inlet, an outlet, and two or more arrays of microposts arranged within the channel between the inlet and the outlet. A first micropost array is arranged in the channel closer to the inlet than a second and subsequent micropost arrays. The second micropost array is arranged in the channel closer to the outlet than the first micropost array. A subsequent micropost array, if any, is arranged in the channel between the second micropost array and the outlet. Microposts in the subsequent array are arranged (i) closer together than microposts in the second micropost array and (ii) microposts in the second array are closer together than microposts in the first array. Structures in the second and subsequent micropost arrays are arranged at a distance apart that enables capture of target entity-secondary entity complexes, but provides sufficient space to allow target entities and secondary entities to flow through and out of the five micropost arrays. A second channel in fluid communication with the first channel and having an inlet, a first outlet, a second outlet, a third outlet, and two rows of filtering structures arranged within the second channel between the inlet and the third outlet. A first row of filtering structures is spaced apart in the second channel from a second row of filtering structures such that spacing between the first row and the second row defines a central portion of the second channel. Structures in the first row and the second row are spaced apart along a direction of flow in the central portion so as to permit non-target entities to flow through spaces between the structures and out the first outlet or the second outlet of the second channel. The first row and the second row are spaced art perpendicular to direction of flow in the central portion. The central portion provides sufficient space to allow target entities and secondary entities to flow through and out of the third outlet of the second channel. A third channel in fluid communication with the second channel and having an inlet, a product outlet, and three or more micropost arrays of structures arranged within the third channel between the inlet and the product outlet Structures in the three or more micropost arrays are arranged at a distance apart to release target entity-secondary entity complexes through and out of the product outlet of the third channel.

In some implementations, the first channel, the second channel, and the third channel are provided in a single substrate.

In some implementations, structures included in the first row and the second row of the second channel are sized to have a length between 650-850 μm along a direction of flow in the central portion. Structures included in the first row and the second row of the second channel are spaced apart between 30-60 along a direction of flow of in the central portion. The central portion has a length between 400-600 μm perpendicular to a direction of flow in the central portion.

In some implementations, a height of the structures included in the first row and the second row is between 100-1000 μm. For example, the height is approximately 350 μm.

In some implementations, the three or more micropost arrays of structures arranged within the third channel includes five micropost arrays of structures. A first array of the five micropost arrays of structures is arranged in the third channel closer to the inlet than a second array of the five micropost arrays of structures. A third array of the five micropost arrays of structures is arranged in the third channel between the first array and the second array. A fourth array of the five micropost arrays of structures is arranged in the third channel between the third array and the second array. A fifth array of the five micropost arrays of structures is arranged in the third channel between the fourth array and the second array. Structures included in the first array are arranged further apart than structures in the second array. Structures included in the third array are separated by gap sizes that are (i) larger than gap sizes between the structures included in the second array, the fourth array, and the fifth array, and (ii) smaller than gap sizes between the structures included in the first array. Structures included in the fourth array are separated by gap sizes that are (i) larger than gap sizes between the structures included in the second array and the fifth array, and (ii) smaller than gap sizes between the structures included in the third array. Structures included in the fifth array are separated by gap sizes that are (i) larger than gap sizes between the structures included in the second array, and (ii) smaller than gap sizes between the structures included in the fourth array.

In some implementations, the structures included in the first array have gap sizes between structures of 95-145 μm. The structures included in the second array have gap sizes between structures of 5-55 μm. The structures included in the third array have gap sizes between structures of 75-125 μm. The structures included in the fourth array have gap sizes between structures of 55-105 μm. The structures included in the fifth array have gap sizes between structures of 25-75 μm.

In some implementations, a height of the structures included in the first array, the second array, the third array, the fourth array, and the fifth array is between 100-1000 μm. For example, the height is approximately 350 μm.

In another general aspect, the disclosure provides methods of extracting oocytes from a sample fluid including cumulus oocyte complexes (COCs). The method includes several steps. At step (a), the methods include capturing one or more COCs, if any, in the sample fluid using a microfluidic device. The microfluidic device includes (i) a size-based capture mechanism in the microfluidic device. The microfluidic device also includes a channel having an inlet, an outlet, and two or more arrays of structures arranged within the channel between the inlet and the outlet. A first array is arranged in the channel closer to the inlet than a second array. Structures in the first array are arranged further apart than structures in the second array. Structures in the second array are configured and arranged at a distance apart that enables capture of COCs, but provides sufficient space to allow cells and/or debris smaller than COCs to flow through and out of the size-based capture mechanism. The microfluidic device also includes (ii) a binding agent that provides a binding interaction with cumulus cells and/or granulosa cells in the COCs within the microfluidic device. The microfluidic device also includes a channel having an internal surface, an inlet, and an outlet. The binding agent is attached to the internal surface and binds specifically or non-specifically to the cumulus cells and/or granulosa cells.

At step (b), the methods include flowing a reagent solution through the microfluidic device to weaken or break a specific binding interaction between the oocytes and the cumulus cells and/or between the oocytes and the granulosa cells, thereby releasing the oocytes from the COCs. At step (c), the methods include capturing a portion of the reagent that includes the oocytes.

In some implementations, the binding agent includes a non-specific binding agent.

In some implementations, the non-specific binding agent includes poly-L-lysine and/or laminin.

In some implementations, the binding agent includes a specific binding agent that specifically binds to cumulus cells and/or granulosa cells.

In some implementations, the specific binding agent includes an antibody that specifically binds to cumulus cells and/or granulosa cells.

In some implementations, the antibody includes a follicle-stimulating hormone receptor (FSHR) antibody, luteinizing hormone choriogonadotropin receptor (LHCGR) antibody, or Anti-Mullerian hormone receptor type 2 (AMHR2) antibody.

In some implementations, the reagent solution includes hyaluronidase.

In some implementations, the method further includes, before step (a), filtering and removing from the sample fluid at least some cells and/or debris smaller than about 50 microns.

In some implementations, the method further includes, after step (b) and before or after step (c), denuding the oocytes from any remaining surrounding cumulus cells and/or granulosa cells.

In some implementations, the method further includes after step (c) or after a denuding step, transferring the oocytes from the reagent solution and concentrating the oocytes into another, different solution.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control.

In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A is a schematic of an example of a system with a microfluidic device configured to use a two-step process for capturing COCs from a FF spiked with COCs extracted from a mouse.

FIG. 1B is a schematic of one implementation of the microfluidic device shown in FIG. 1A. FIG. 1B shows examples of a first module with three micropost arrays of structures and a second module with two channels implemented on a single microfluidic chip.

FIGS. 2A and 2B are schematics of a first module of the microfluidic device shown in FIG. 1A. FIG. 2A shows a detailed view of a channel of the first module with micropost arrays of structures. FIG. 2B shows examples of dimensions (e.g., 1000 μm, 500 μm, 250 μm, 125 μm) of the spacing of gaps between adjacent structures in micropost arrays of the channel shown in FIG. 2A.

FIGS. 3A-3C are schematics of a second module of the microfluidic device shown in FIG. 1A. FIG. 3A shows a detailed view of two channels of the second module. A first channel includes rows of filtering structures and a second channel includes micropost arrays of structures. FIG. 3B shows examples of dimensions for spaces between the filtering structures of the first channel. FIG. 3C shows examples of dimensions (e.g., 120 μm, 100 μm, 80 μm, 50 μm, 30 μm) of the spacing of gaps between adjacent structures in micropost arrays of the second channel shown in FIG. 3A.

FIG. 4 is a schematic of an integrated microfluidic system that can capture oocytes from a human follicular fluid aspirate (FFA) sample.

FIGS. 5A and 5B are schematics of a microfluidic device of the system shown in FIG. 4 used for COC isolation.

FIG. 6 is a schematic of a third-stage microfluidic device of the system shown in FIG. 4.

FIG. 7 is a table with results of a preliminary capture experiment conducted using the microfluidic device shown in FIG. 1A.

FIGS. 8A to 8H are a series of representations of microscope images of the channel shown in FIG. 2A that were collected during capture experiments using the microfluidic device shown in FIG. 1A.

FIGS. 9A to 9E are a series of representations of microscope images of the channel shown in FIG. 3C that were collected during capture experiments using the microfluidic device shown in FIG. 1A.

Like reference symbols in the various drawings indicate like elements.

### DETAILED DESCRIPTION

TE isolation generally involves a two-step process in which TE-SE complexes are initially captured (i.e., TE-SE complexes are separated from biological fluid) and then TEs are specifically released from the captured TE-SE complexes. TE-SE complexes can be captured using size-based approaches or using specific and/or non-specific interactions between SEs and ESs. TE release can be achieved using biochemical means (e.g., using disassociating enzymes and/or inhibitors) or using physical properties of fluid flow (e.g., flow velocity and/or shear rate).

As described herein, the formation of TE-SE complexes in biological fluids provide opportunities to develop isolation methods, such as using SE surface markers to selectively capture TEs, size-based capture based on the larger size of TE-SE complexes, or using TE-SE interactions to promote specific release of TEs. Thus, the present disclosure features systems and methods for isolating TEs found in TE-SE complexes within a biological fluid by exploiting interactions between TEs, SEs, and engineered surfaces. TE isolation generally involves a two-step process in which TE-SE complexes are initially captured (i.e., TE-SE complexes are separated from biological fluid) and then TEs are specifically released from the captured TE-SE complexes. Such TE-SE complexes can be captured using size-based approaches or using specific and/or non-specific interactions between SEs and an engineered surface. TE release can be achieved using biochemical means (e.g., using disassociating enzymes or inhibitors) or using physical properties of fluid flow (e.g., flow velocity or shear rate). Various techniques of TE-SE complex capture and specific TE release are thereby contemplated within this disclosure.

The systems and techniques described herein enable various processes to isolate TEs from fluid samples. The first isolation process involves non-specific binding between SEs and module 110A followed by specific release of TEs (such as oocytes and/or COCs) from the SEs. The system can be coated with a binding agent to provide specific binding (e.g., using an antibody) or non-specific binding (e.g., using a polymer) of entities found in the biological fluid sample, such as FF (e.g., poly-L-lysine, laminin, Corning Cell-Tak®). In this first step, a biological fluid sample, such as FF, is passed through a microfluidic device, which enables attachment of COCs and other entities to a surface. In a second stage, specific release of TEs, such as un-denuded (or partially denuded) COCs, is achieved by passing a release agent fluid (e.g., hyaluronidase) that specifically weakens interactions between SEs and/or interactions between TEs and SEs.

A second isolation process involves specific binding between SEs and a microfluidic device and specific release of TEs from SEs. A microfluidic device is coated with an agent (e.g., polymer, antibody) for specific binding of SEs, such as cumulus cells (CCs) or granulosa cells (CGs). Examples of agents used in this process include follicle-stimulating hormone receptor (FSHR) antibody, luteinizing hormone/choriogonadotropin receptor LHCGR) antibody, anti-Mullerian hormone receptor type 2 (AMHR2) antibody. In a first stage of this process, a biological fluid sample, e.g., FF, is passed through the microfluidic device, which enables attachment of TEs, such as COCs and other free CGs, to the device surface. In a second stage, specific release of TEs, such as un-denuded (or partially denuded) COCs, is achieved by passing a release agent fluid (e.g., hyaluronidase) that specifically weakens interactions between SEs and/or interactions between TEs and SEs.

A third isolation process involves specific binding between SEs (e.g., CGs) and the microfluidic device, and nonspecific release of TEs from SEs. The microfluidic device is coated with an agent (e.g., polymer, antibody) for specific binding of CGs. Examples of agents used in this process include follicle-stimulating hormone receptor (FSHR) antibody, luteinizing hormone/choriogonadotropin receptor LHCGR) antibody, anti-Mullerian hormone receptor type 2 (AMHR2) antibody. In a first stage of this process, FF is passed through the microfluidic device, which enables attachment of COCs and other free CGs to the device surface. In a second stage, specific release of un-denuded (or partially denuded) COCs is achieved by passing a fluid (e.g., hyaluronidase) at a high shear rate that enables release of large bodies of TEs attached to the surface due to increased fluidic drag, while smaller free entities remain attached to the device surface. In some embodiments, TEs can be released by disturbing the binding between SEs within the TE-SE complexes.

In some embodiments, the second and third processes discussed herein are adjusted such that magnetic beads are used for the initial capture of COCs as an alternative to ESs. Magnetic beads are functionalized for specific binding to CGs on COCs. A magnetic field is then applied to sort COCs in a microfluidic device. Oocytes are then released from COCs by specific breaking of interactions between oocytes and CGs (e.g., using hyaluronidase) or nonspecific breaking of interactions between oocytes and CGs (e.g., using high shear flow).

### A. Target Entity Capture Device

### a. Overview

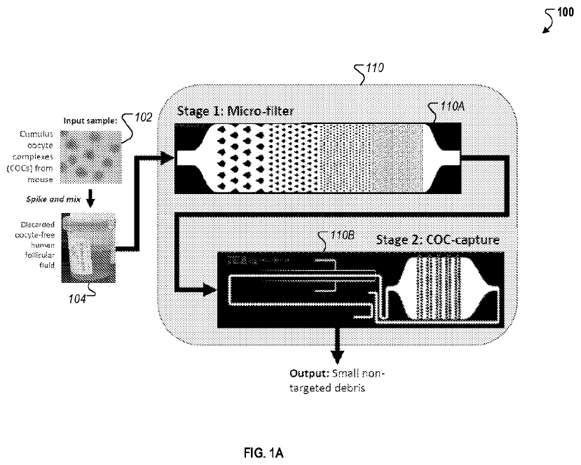

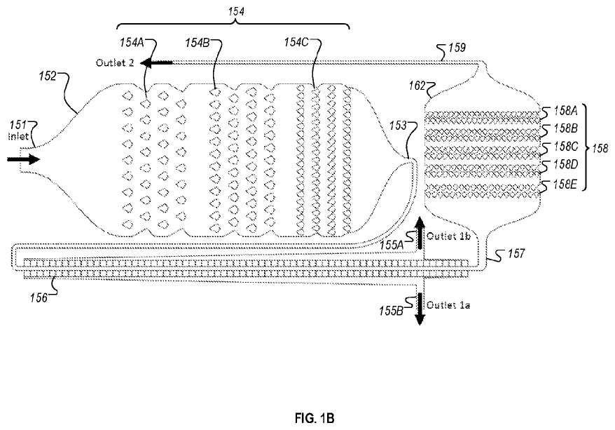

FIG. 1A is a schematic of an example of a system 100 that provides a two-stage process for capturing TEs 102 (e.g., COCs) from a human sample 104 (e.g., FFA sample) using a microfluidic device or “chip” 110. System 100 enables targeting of different interactions in each stage (capture, release) while still enabling entity-specific isolation of TEs 102. This technique allows non-specific interactions to be used in either capture (stage one) or release (stage two) stages, since the combination yields an overall entity-specific isolation paradigm. Non-specific interactions between SEs and ES can be used for high efficiency capture of TE-SE complexes, and a specific interaction between TEs 102 and SEs can be used for releasing TEs 102 while enabling entity-specific TE 102 isolation. In some embodiments, the techniques described throughout can be applied to capture other entities, such as circulating tumor cell clusters (CTCC).

FIG. 1B is a schematic of one implementation of the microfluidic device 110 shown in FIG. 1A. In this implementation, modules 110A and 110B are combined on a single microfluidic chip. In other implementations of microfluidic device 110, modules 110A and 110B may be provided on separate microfluidic chips. For example, module 110A may be on a first microfluidic chip that is in fluidic communication with a second microfluidic chip that includes module 110B. In each of these various implementations, the device 110 provides a two-stage process for capturing and isolating TEs, as described herein.

Module 110A is used in the first stage of the two-stage technique discussed herein. Module 110B is configured to capture larger non-target entities (e.g., tissue debris) in a sample (e.g., FFA sample) while also allowing TEs (e.g., COCs) to be captured at the smaller filters or in a subsequent step. Module 110A includes a channel 152 with different micropost arrays of 154A, 154B, and 154C (collectively which make up a plurality of micropost arrays 154) between an inlet 151 and an outlet 153. The sizes of structures within the micropost arrays 154 gradually increases in a longitudinal direction of the channel 152.

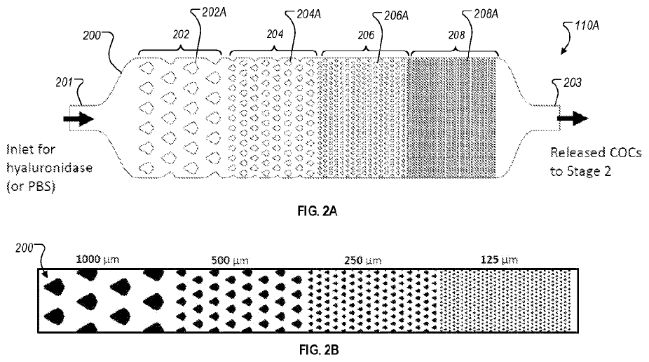

Module 110B is used in the second stage of the two-stage technique discussed herein. Module 110B includes a first channel 156 configured to reduce initial volume of fluid entering channel 300A via inlet 153 from channel 152. This results in approximately 80%-99% of fluid flow being directed towards side outlets 155A, 155B to reduce flow velocity of fluid exiting outlet 157 and entering the capture stage in channel 162. Reduction in fluid flow is accomplished with a plurality of micropost arrays 158 (e.g., micropost arrays 158A, 158B, 158C, 158D, 158E). Target entities are captured and then extracted via outlet 159. Various implementations and configurations of device 110 are contemplated within this disclosure. Amongst these implementations, the device 110 is configured to provide a two-stage process for capturing and releasing TEs 102 from a sample 104. However, the configurations of modules 110A, 110B (structures within them) of device 110 may vary depending on the implementation needs associated with a specific type of TE. The number of micropost arrays within channel 152 may vary depending on capture requirements of a TE. For example, FIG. 1B shows on implementation in which a channel in module 110A has three micropost arrays of micropost structures (154A, 154B, 154C), while FIG. 2A shows another implementation in which a channel in module 110A has four micropost arrays (202, 204, 206, 208).

Additionally, the gap sizes between structures within micropost arrays of module 110A may also vary depending on capture requirements of a TE. Exemplary gap sizes include approximately 700 μm to approximately 1200 μm, e.g., about 900 μm to about 1000 μm, approximately 350 μm to approximately 650 μm, e.g., about 400 μm to about 500 μm, approximately 150 μm to approximately 300 μm, e.g., about 150 μm to about 250 μm, approximately 75 μm to approximately 150 μm, e.g., about 100 μm to about 125 μm.

In some implementations, device 110 is architected to provide size-based capture of COCs and specific release of COCs by enzymatically disassociating secondary entities. In this architecture, COCs are captured based on their size using a filter, and released by sacrificing a portion of the CG layer via hyaluronidase treatment and increased flow velocity. Losing a sacrificial layer of CGs can reduce the effective size of a COC (TE-SE complex) and thereby enable release from a size-based filter. This technique can be achieved using a microfluidic system that captures COCs using a two-step process.

In other implementations, device 110 is configured to improve development of integrated microfluidic devices for COC isolation from biological fluids. Though some oocyte denudation microfluidic devices have been proposed to extract CCs from COCs suspended in a small volume of buffer solution, such oocyte denudation microfluidic devices often have various limitations. For example, oocyte denudation microfluidic devices are not often suitable for use with follicular fluid aspirate (FFA), because FFA contains contaminants such as large tissue debris and blood clots that do not respond to hyaluronidase treatment and can impede capture performance or produce clogging within the devices. Additionally, smaller tissue debris (e.g., blood cells) may also be captured with denuded oocytes, thereby reducing isolation purity. Given these features, to isolate COCs from FFA, removal of tissue debris upstream of a denudation microfluidic device is desired. The systems and techniques disclosed herein achieve this through a three-phase process discussed below in reference to FIGS. 4-7.

In some implementations, the device 110 is configured to various mammalian fertilization applications. For example, techniques can be used to isolate COCs from fluids from fluid samples extracted from mammals including humans, mice, rats, rabbits, monkeys, sheep, goats, cats, dogs, horses, cows, and pigs, among others. In some applications, the sample fluid is obtained by sacrificing a mammal and manually dissecting the ovarian/follicular tissue. In such applications, the sample fluid is a dissected ovarian tissue sample. In other applications, the sample fluid is a dissected ovarian tissue sample obtained from a human that is undergoing treatment (e.g., cancer treatment) and may have their ovaries/follicles removed for long term oocyte banking.

b. TE Capture Techniques

To increase fluid throughput, multiple microfluidic channels and regions containing TE capture zones can be formed on the microfluidic device. For example, as shown in FIG. 1A, microfluidic device 110 can be fabricated to include multiple microfluidic channels that lead to different regions on the device having TE capture zones. A fluid sample is fed into the device 110 using input tubing. Device 110 also includes multiple microfluidic channels fluidly coupled to the output of the TE capture zones. The fluid is removed from the device using an output tubing.

The height/thickness of the structures in the various channels disclosed herein (e.g., channels 152, 156, 162, 200, 300A, 300B), as measured from the uppermost surface of the substrate, may be in the range of about 10 μm to about 1,000 μm including, for example, about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. Other heights can be used as well. The surface area of the structures, as measured along a plane parallel to the uppermost surface of the substrate may be in the range of about 78 μm² to 0.125 mm² including, for example, about 200 μm², about 500 μm², about 1000 μm², about 5000 μm², about 0.01 mm², about 0.05 mm², or about 0.1 mm². Other areas can be used as well.

In some implementations, the device 110 is fabricated such that the heights of structures in the various channels are the same (e.g., about 10 μm, about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. In such implementations, heights of the structures is the same in each stage of the two-stage systems discussed herein. In other implementations, the device 110 is fabricated such that heights of structures are different in each channel. For example, in these other implementations, structures in channel 152 may have a first height, structures in channel 156 may have a second height, and structures in channel 162 may have a third height, where the first height, the second height, and the third height are each different from one another. Different configurations of varying heights among the structures of each channel in the range of about 10 μm to about 1,000 μm are also contemplated.

The rate at which the sample fluid is passed through the TE capture zones is relatively slow compared to the rate that the washing fluid is applied in reverse to release trapped clusters. The slower flow rate is used for the fluid sample so that the shear forces on the TEs are not so high that the forces would push the clusters through the output flow paths of the TE capture zones. For releasing the trapped particles, however, a much higher flow rate is used to wash away individual particles that may have become weakly bound to the device walls and to help release clusters that may also have become weakly bound to the device walls. For instance, the total volume flow of a fluid sample through a device containing TE capture zones during a capture stage can be, e.g., in the range of about 0.1 ml/hr to about 3 ml/hr, whereas the total volume flow of a buffer solution through the device when releasing trapped clusters can be, e.g., in the range of 20 ml/hr to about 250 ml/hr, e.g., 20, 30, 40, 50, 60, 70, 80, 90, 100, 110, 120, 130, 140, 150, 160, 170, 180, 190, 200, 210, 220, 230, 240, or 250 ml/hr.

The total volume through the device can also be increased or decreased based on the overall size and/or number of flow paths of the device. The rate at which fluid flows through each of the TE capture zones is determined by dividing the total fluid flow rate by the number of TE capture zones in the microfluidic device. For example, assuming a particular microfluidic device includes 4000 TE capture zones, and the overall flow rate through the device is 2.5 ml/hr during the cluster trapping stage, then the average flow rate through each TE capture zone is about 0.625 μl/hr. The flow rate of a fluid sample through TE capture zones during the trapping stage can be in the range of, for example, about 0.1 μl/hr to about 10 μl/hr including 0.5 μl/hr, 1 μl/hr, 2 μl/hr, 4 μl/hr, 6 μl/hr or 8 μl/hr. Other flow rates for the fluid sample during the trapping stage are also possible. The flow rates of the fluid sample through the TE capture zones also correspond to a shear force. For example, for each TE capture zone, the shear flow of the fluid sample during the “capture” stage in each of the output flow paths may be less than about 50 s⁻¹ including, e.g., 40 s⁻¹, 30 s⁻¹, 20 s⁻¹, 10 s⁻¹, 10 s⁻¹, or 0.5 s⁻¹. Other shear flow values also may be used.

Due to the relatively slow flow of fluid in the device, gravitational forces can, in some implementations, cause particles from the fluid sample to accumulate near the interface with the substrate, causing clogging of the device. To avoid such clogging, the device can be placed on its side so that the gravitational force is in the direction of the output of the microfluidic device, instead of toward the substrate.

As explained above, microfluidic devices containing TE capture zones can be used to trap and subsequently isolate TE from fluid samples without requiring the TE to bind to a surface of the device. In some cases, however, other undesired particles bind, either specifically or non-specifically, to regions of the microfluidic device, thus lowering the purity of the isolated TE. Alternatively, or in addition, the TE themselves may non-specifically bind to portions of the microfluidic device, making it more difficult to release the trapped clusters upon passing a solution in the reverse direction to the direction of the initial fluid sample flow. Examples of microfluidic devices containing TE capture zones are disclosed in U.S. Pat. Nos. 10,150,116 and 10,786,817, each of which is incorporated herein by reference.

There are several techniques that can be used to avoid or inhibit this non-specific binding. For example, one technique for limiting the amount of undesired binding of particles to a microfluidic device surface includes lowering the temperature of the solution and the particles contained within the solution. In the case that the particles within the fluid sample are cells, lower temperatures (relative to ambient, e.g., room temperature) lead to a reduction in cell-to-surface bond formation. Accordingly, with fewer bonds being formed, fewer cells bind to the device surface. Thus, in a microfluidic device configured to trap and isolate a specific type or types of cells, the number of undesired cells that inadvertently bind to the device surface can be reduced, thus increasing isolation purity of desired cells.

i. Large Tissue/Debris Removal From TE-SE Clusters

FIG. 2A is a schematic of a channel of module 110A. Module 110A is configured to capture larger non-target entities (e.g., tissue debris) in the sample 104 (e.g., FFA sample) while also allowing TEs (e.g., COCs) to be captured at the smaller filters or in a subsequent step. Module 110A includes a channel 200 with different micropost arrays of 202, 204, 206, and 208 between an inlet 201 and an outlet 203. The sizes of structures within the micropost arrays gradually increases in a longitudinal direction of the channel 200.

In one example, which is shown in FIG. 2B, structures 202A are arranged to have gap sizes that are approximately 1000 μm, structures 204A are arranged to have gap sizes that are 500 μm, structures 206A are arranged to have gap sizes that are 250 μm, and structures 208A are arranged to have gap sizes that are 125 μm. Other configurations and dimensions are also contemplated with this disclosure. For example, structures 202A can be arranged to have gap sizes between structures of approximately 700 μm to approximately 1200 μm, e.g., about 900 μm to about 1000 μm. The structures 204A can be arranged to have gap sizes between structures of approximately 350 μm to approximately 650 μm, e.g., about 400 μm to about 500 μm. The structures 206A can be arranged to have gap sizes between structures of approximately 150 μm to approximately 300 μm, e.g., about 150 μm to about 250 μm. The structures 208A can be arranged to have gap sizes between structures of approximately 75 μm to approximately 150 μm, e.g., about 100 μm to about 125 μm.

ii. Small Tissue Debris Removal and TE-SE Clusters Capture

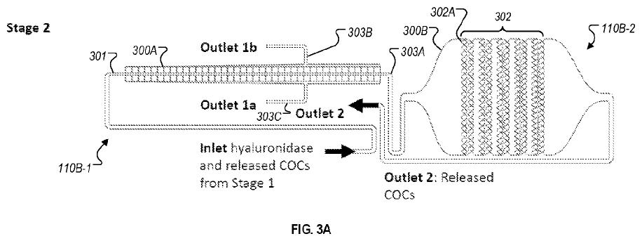

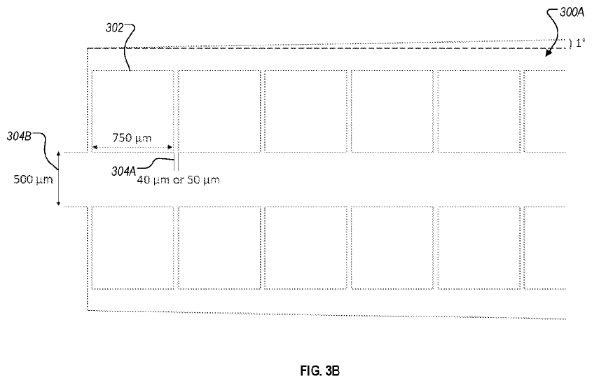

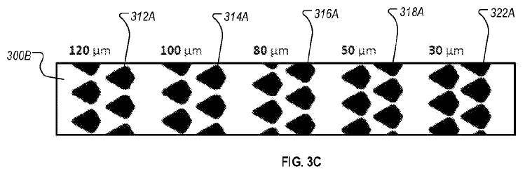

FIG. 3A is a schematic of two channels of module 110B. Module 110B is configured to reduce initial volume of fluid entering channel 300A via inlet 301 from channel 200 (shown in FIGS. 2A, 2B). This results in approximately 80%-99% of fluid flow being directed towards side outlets 303B, 303C to reduce flow velocity of fluid exiting outlet 303A and entering the capture stage in channel 300B. Reduction in fluid flow is accomplished with structures (e.g., side filters), as shown in FIG. 3B.

FIG. 3B shows examples of dimensions for spaces associated with the filtering structures 302 within channel 300A. Structures 302 are arranged in two rows extending in a longitudinal direction of channel 300A (top row, bottom row). Each of the structures 302 are horizontally spaced apart within each row by spaces 304A. The rows are also vertically spaced apart in the channel 300A by space 304B and defines a central portion of channel 300A. In some implementations, spaces 304A are sized to be between 40-50 μm and space 304B is sized to be 500 μm. Spaces 304 permit siphoning of a percentage of fluid flowing through channel 300B (e.g., approximately 1-2%) to ensure that COC continue to flow through a central portion of the channel. Small debris within the fluid (e.g., red blood cells) are permitted to flow through spaces 304 and thereby allow removal of small debris without removing COCs (which instead flow through the central portion).

The size and/or arrangement of structures 302 within the two rows extending in a longitudinal direction of channel 300A (top row, bottom row) may vary depending on capture requirements of a TE. For example, the size of spaces 304A can be between 30-60 μm along a direction of flow in a central portion of channel 300A. Additionally, the length of structures 302 can be between 650-850 um along a direction of flow in a central portion of channel 300A. Further, space 304B can have a length between 400-600 μm perpendicular to a direction of flow in the central portion of channel 300A.

FIG. 3C shows examples of dimensions of the micropost arrays of structures 302 of channel 300B. As shown, structures 312A are arranged to have a gap size of about 120 μm, structures 314A are arranged to have a gap size of about 100 μm, structures 316A are arranged to have a gap size of about 80 μm, structures 318A are arranged to have a gap size of about 50 μm, and structures 322A are arranged to have a gap size of about 30 μm. Other configurations and dimensions are also contemplated with this disclosure.

Additionally, the gap sizes between structures within the micropost arrays of channel 300B may vary depending on release requirements of a TE. Exemplary gap sizes include approximately 95 μm to approximately 145 μm, e.g., about 100 μm to about 120 μm, approximately 75 μm to approximately 125 μm, e.g., about 90 μm to about 100 μm, approximately 55 μm to approximately 105 μm, e.g., about 70 μm to about 80 μm, approximately 25 μm to approximately 75 μm, e.g., about 40 μm to about 50 μm. approximately 5 μm to approximately 55 μm, e.g., about 20 μm to about 30 μm.

c. Device Fabrication

Microfluidic devices described herein (e.g., microfluidic device 110) can be manufactured using various soft lithography methods. As one example, a mold defining the features of the device 100 is obtained. The mold can be formed by applying and sequentially patterning two layers of photoresist (e.g., SU8, Microchem, Newton, Mass.) on a silicon wafer using two photolithography masks. The masks can contain features that define the different aspects of the device 100, such as the input microfluidic channels, the TE capture zones, and the output microfluidic channels. The wafer with the patterned photoresist then may be used as a master mold to form the microfluidic parts. A polymer (e.g., polydimethylsiloxane (PDMS), polymethylmethacrylate (PMMA), or polycarbonate (PC)) solution then is applied to the master mold and cured (e.g., by heating).

After curing, the polymer layer solidifies and can be peeled off the master mold. The solidified polymer layer includes recesses corresponding to the fluid channels and fluid pathways of the TE capture zones. The polymer layer then is bonded to a substrate such as a glass slide. For example, a bottom surface of the polymer layer can be plasma treated to enhance the bonding properties of the polymer. The plasma treated polymer layer then may be placed on the glass slide and heated to induce bonding. After bonding the polymer to the glass slide, a cover slide (e.g., a glass slide) may be bonded to a top of the polymer layer to enclose the microfluidic channels and TE capture zones. The surface of the polymer layer contacting the cover slide may also be plasma treated before bonding to the cover.

In the example discussed above, microfluidic device 110 includes a substrate layer of glass, a polymer layer defining the microfluidic channels and the TE capture zones, and a cover layer made of glass. In other implementations, the substrate layer and/or the cover layer can be polymer substrates or other similar materials.

The foregoing technique is just one example of a fabrication method for the microfluidic device. Other techniques may be used instead. For example, techniques such as hot embossing, LIGA (which stands for Lithographie, Galvanoformung, und Abformung, which is German for Lithography, Electroplating, and Molding), or injection molding may be used to fabricate one or more layers of the microfluidic device including the TE capture zones.

d. Applications

There is an ever increasing need in biological research, for example, for more accurate and efficient methods to manipulate and separate target particle and cell populations. Disciplines ranging from immunology and cancer medicine to stem cell biology are highly dependent on the identification of uncontaminated populations of particular particle and cell subsets for detailed characterization. Clinically, microbiologists routinely isolate bacterial cells and white blood cell subsets for diagnostic purposes. Tumor antigen-specific regulatory T cells can be discovered in the circulating blood of cancer patients, presenting a new potential target for immunotherapy of metastatic melanoma. Environmental sensing requires surveillance of water, food and beverage processing for specific bacterial cell contamination. Vaccine developers work largely with antigen-specific T-lymphocytes, rare cells which may differ from one another by no more than a single amino acid in a peptide fragment presented on the cell surface.

In these different applications a common problem is presented: the need to isolate, separate and characterize subpopulations of cells present within heterogeneous, complex fluids. During the processing of these samples, the target cell population are handled with gentle care, preventing alteration of the cell's physiological state to allow for subsequent expression profiling and molecular studies. Moreover, the cells of interest may be present at extremely low frequencies-often less than 1 cell in 10,000,000 cells, for circulating tumor cells or disease-specific T lymphocytes, increasing the complexity of the challenge.

The devices containing TE capture zones described herein can be used as a means of isolating cells and cell clusters found in fluid samples for the above-mentioned research and analysis. For example, in some implementations, a blood sample extracted from a patient may or may not contain a number of circulating tumor cells (CTCs) or CTC clusters (CTCCs), which can be indicative of the occurrence of cancer metastasis in the patient. A user interested in identifying the presence of the CTC clusters can use the microfluidic device to isolate CTC clusters present in the blood sample from individual cells (e.g., individual white blood cells or individual red blood cells) that are not part of a cluster. Once the cell clusters have been isolated, a user may then perform an analysis on the isolated clusters (e.g., count the number of CTC clusters present in the blood sample to diagnose the patient, to study disease progression, or to study the response of the patient to a treatment). The devices described herein are not limited to uses involving isolation of CTC clusters and can be used in a wide range of applications requiring enumerating, sorting, concentrating and ordering of TE or removing undesired TE from fluid samples.

The systems and methods described herein thus provide a manner in which rare cells or clusters of rare cells, such as CTC clusters, can be sorted, separated, enumerated, and analyzed continuously and at high rates. Whether a particular cell cluster is a rare cell cluster can be viewed in at least two different ways. In a first manner of characterizing a cell cluster as rare, the rare cell cluster can be said to be any cell that does not naturally occur as a significant fraction of a given sample. For example, for human or mammalian blood, a rare cell cluster may be any cell cluster other than a subject's normal blood cell (such as a non-cancerous red blood cell and a non-cancerous white blood cell). In this view, cancer or other cells present in the blood would be considered rare cells.

In a second manner of characterizing a cell cluster as rare might take into account the frequency with which that cell cluster appears in a sample. For example, a rare cell cluster may be a cell cluster that appears at a frequency of approximately 1 to 50 cells per ml of blood. Alternatively, rare cell cluster frequency within a given population containing non-rare cells can include, but is not limited to, frequencies of less than about 1 cell cluster in 100 cells; 1 cell cluster in 1,000 cells; 1 cell cluster in 10,000 cells; 1 cell cluster in 100,000 cells; 1 cell cluster in 1,000,000 cells; 1 cell cluster in 10,000,000 cells; 1 cell cluster in 100,000,000 cells; or 1 cell cluster in 1,000,000,000 cells.

### B. Integrated Microfluidic Systems

### a. Overview

FIG. 4 is a schematic of an integrated microfluidic system 400 that can be configured to capture and release TE, for example, oocytes or COCs, from a human sample 401, such as an FFA sample. During step 410, human sample 401 is flowed through microfluidic devices 402, 404, and 406. TEs within the human sample 401 are captured in a channel of device 402 and non-target entities and tissue debris are filtered out via waste outlets. During step 420, a buffer solution 403, for example, a hyaluronidase solution, is flowed through devices 402, 404, and 406. This results in TE captured in device 402 being subsequently released for downstream processing. During 430, an exchange solution 405 is flowed through device 406 to provide solution exchange to selectively extract TE into a product outlet 407 and discharge buffer solution 403 through a waste outlet.

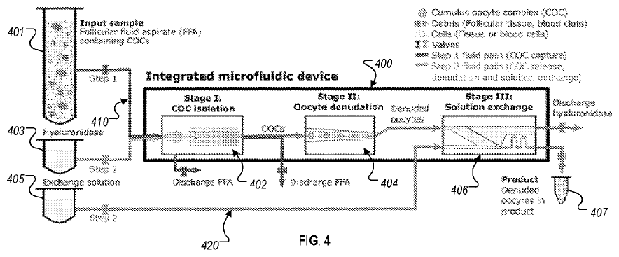

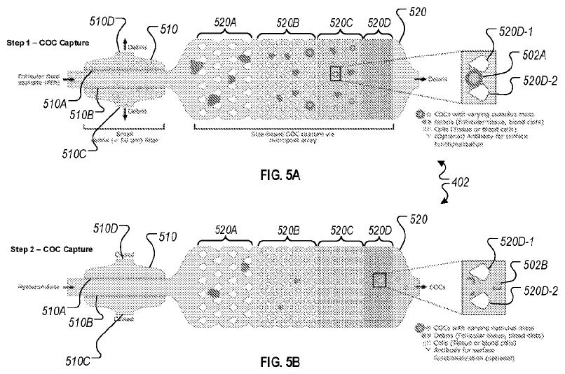

In one example, system 400 is configured to generate intracytoplasmic sperm injection (ICSI) or freeze-ready oocytes (or COCs) from a patient sample. This is accomplished using the procedure discussed above and shown in FIG. 4. In general, COCs are isolated (capture, release) from FFA sample in device 402 (shown in FIG. 5A) and then released from device 402 (shown in FIG. 5B). Captured COCs are denudated in device 404 (shown in FIG. 6). Denuded COC are then extracted to device 406 and collected in product output 407 (shown in FIG. 7).

In the example discussed above, device 402 (which, in some instances, can be the microfluidic device 110 shown in FIG. 1A) is used to remove non-target entities and tissue debris from the FFA sample and captures COCs. This allows to intrinsic variabilities between different FFA samples to be eliminated and thereby enables steady release of captured COCs for denudation. Once the FFA sample is processed, input to device 402 is switched to hyaluronidase, which allows captured COCs to be released for denudation.

In this example, device 404 is used to remove CC from isolated COC while preserving oocyte viability and functionality. Cumulus-free COCs are then flowed through device 406 and then transferred to product output 407 (e.g., media, cryopreservation holding solution) depending on a clinical goal. COCs can also concentrated into a smaller product volume, while further removing any carryover debris.

In some instances, fluid flows through device 404 based on pump-flow action. As the liquid sample flows through device 404, the COCs pass through the several expansion units, smooth constriction units, and jagged constriction units of device 404. In general, the expansion units are designed to cause the COCs to tumble, which helps as much of the surface of the COCs as possible to contact the inner walls of the following constriction channels. The smooth constriction units facilitate the removal of the bulky, loosely attached cumulus cells of the COCs, and the jagged constriction units facilitate the removal of the corona radiata of the COCs. The constriction unit includes jagged teeth angled towards the flow of the COCs. As the COCs traverses the jagged constriction unit, the jagged teeth apply shear stress on the COC, thereby facilitating the denudation of the oocyte in the COCs. As the COCs make their way through the device 404, the COC become more and more denuded. For example, COCs can flow through three separate jagged constriction units. COCs become more and more denuded as they traverse through each jagged constriction unit. When the COCs 170 pass through the final series of jagged constriction units, the oocytes have become mostly or completely denuded.

Using the procedure discussed above, an oocyte preparation process can be completed in capture and release steps. In the capture step, only the FFA input will flow into the system 400, and COCs will be captured in device 402 (shown in FIG. FIG. 4 as path 410). During the release step, hyaluronidase and buffer solutions are open, and COCs are released from device 402 and denuded in device 404. The denuded COCs are then concentrated into a small volume of product solution in device 406 (shown in FIG. 4 as path 420).

b. TE Capture Techniques
i. TE-SE Complex Isolation

FIGS. 5A and 5B show detailed views of the release and release stages of microfluidic device 402. In a first step (shown in FIG. 5A), non-target entities and tissue debris is filtered out. In this step, a sample fluid is flowed through chamber 510, which includes filters 510A and 510B and outlets 510C and 510D. As fluid flows through chamber 510, small debris (e.g., less than 50 um in diameter) are able to pass filters 510A and 510B and exit channel 510 through outlets 510C and 510D, which act as waste outlets. The sample is then flowed through chamber 520, which includes micropost arrays of structures 520A, 520B, 520C, and 520D. The micropost arrays 520A-D can be sized and structured to provide size-based TE capture via the structures. For example, as shown in FIG. 5A, structures in each micropost or microstructure array are sized to be progressively smaller from the inlet to the outlet to promote capture of different-sized entities within channel 520. The right side of this figure shows how TE 502A is captured between structures 520D-1 and 520D-2 within micropost or microstructure array 520C.

In a second step (shown in FIG. 5B), TEs are captured and subsequently released for downstream processing. In this step, outlets 510C and 510D are closed and before a reagent is flowed through chamber 510. This prevents fluid from exiting chamber 510 via outlets 510C and 510D and thereby results in fluid mostly flowing into chamber 520 from chamber 510. TEs captured in chamber 520 in the first step can be released from the micropost arrays 520A-D by enzymatically detaching layers of TE-SE complexes. By enzymatically weakening such interactions, and the assistance of shear flow provided by the reagent, TEs will be released from chamber 520. In some implementations, the release mechanisms disclosed herein can also target binding interactions between SEs, which also results in TE release. For example, in addition to targeting interactions between an oocyte and cumulus/granulosa cells, the release mechanisms can also target interactions between cells within the cumulus tissue itself.

In one example, device 402 is used to capture TEs representing COCs from a fluid sample representing FFA. In this example, COCs with varying cumulus mass are captured in the first stage shown in FIG. 5A. As FFA is flowed through chamber 510, small debris, such as tissue or RBCs, are able to pass through filters 510A and 510B and exit via waste outlets 510A and 510B. Another portion of the FFA sample then flows into chamber 520. The arrangement of micropost arrays 520A-D within chamber 520 provides a size-based COC capture technique. For example, large debris, such as follicular tissue and blood clots are captured in micropost array 520A, while smaller debris are subsequently captured in micropost arrays 520B-520D. COCs are also captured in these micropost arrays. In the example shown in FIG. 5A, two COCs are captured in micropost array 520B, three COCs are captured in micropost array 520C, and five COCs are captured in micropost array 520D. In some embodiments, array structures are functionalized with an entity that binds with surface antigens of COCs, as shown with the capture of COC 502A between array structures 520D-1 and 520D-2 within micropost array 520C.

In the example discussed above, the reagent used in the second step (FIG. 5B) is hyaluronidase, which enable release of captured COCs from chamber 520 by enzymatically detaching a layer of CCs from the oocyte. Hyaluronidase is a clinically established enzyme routinely used for oocyte denudation. By enzymatically weakening the interactions between the COCs and CCs, and the assistance of the shearing flow, COCs will be released by losing a layer of CCs on the microfluidic surface (shown in FIG. 5B).

ii. Oocyte Denudation

As discussed above, in some examples, system 400 is used to capture TEs, such as COCs, from a fluid sample, such as a mammalian FF sample. In such examples, captured COCs are denudated before release from the system 400 using device 404 (shown in FIG. 4). Device 404 is configured remove CCs from COCs extracted from a subject, e.g., a mammalian subject, such as a human, primate, monkey, horse, cow, goat, rat, mouse, etc. As discussed herein, a majority of the FF and debris are removed in device 402 and COCs in hyaluronidase solution for are released to device 404 for denudation.

Device 404 can be configured for denuding oocytes from surrounding cumulus and corona cells with broad applications in the field of assisted human reproduction. At a general level, device 404 includes at least one channel having one or more stages, each stage having repeating constriction units and expansion units. A liquid sample, e.g., a raw FF sample, containing COCs can be injected into an inlet of system 400, e.g., at a continuous flow rate. The flow rate causes COCs to traverse through the channel of the system. The constriction units and the expansion units work together to facilitate the denudation of the oocytes. For example, the constriction units include surface features, such as smooth or jagged inner surfaces, for stripping or peeling the outer cells from the COCs. The expansion units promote tumbling of the COCs as they make their way through the system. System 400 can be configured in such a manner that a continuous flow of COCs is achieved. An example of systems for denuding oocytes are disclosed in US Patent Application Publication No. US2021/0161635, which is incorporated herein by reference.

Device 404 can also denude oocytes from the surrounding cumulus cells while retrieving COCs from large volumes of liquid samples that may contain various sizes of debris. In some embodiments, an enzyme specific to cumulus cells, e.g., hyaluronidase, is added to a sample, e.g., a FF sample, to loosen COCs, but not other debris, and then the liquid sample is flowed through the systems described herein. The large debris, coagulated blood, etc. are captured in the channels and/or coarse filter upstream, while the COCs get denuded and go through the channels. This way the system 400 removes the large debris while denuding the COCs so oocytes are collected at the exit. This enables several capabilities: (1) automatic denudation of oocytes from surrounding cumulus cells treated with an enzyme, e.g., hyaluronidase, to loosen the cumulus cells from the oocyte; (2) the ability to process a large volume of fluid suspension, such as FF, in a single operation without clogging; (3) selectively trap oversized blood clots and tissue debris; and (4) minimize human intervention with reduced processing time and improved standardization. In particular, the multi-channel systems capture large debris, coagulated blood, etc. in the coarse filters upstream and/or in the channels, while the COCs are denuded and can pass through the channels.

iii. Solution Exchange and Concentration

FIG. 6 shows a detailed view of device 406 of system 400. Device 406 includes a chamber 610 with a sorting filter 602, inlets 620A and 620B, and outlets 630A and 630B. In general, device 406 can be configured to provide both fluid extraction and inertial lift forces by controlling the geometry and dimensions of chamber 610 for the purpose of sorting and/or shifting TEs within or among fluids that enter chamber 610 via inlets 610A and 610B. In particular, through fluid extraction and inertial lift forces, device 406 may be used to transfer fluids to and across different fluidic channels of the device, without an accompanying shift of particles, such that the particles may be indirectly transferred to another fluid. In some embodiments, this can be used to manipulate not only the transfer of fluids across micro-channels but also the position of particles suspended within a fluid sample through the shifting of the particles across fluid streamlines.

In addition to shifting particles between fluids, the combination of fluid extraction and inertial lift force enables a number of different ways of manipulating fluids and particles. For example, in some embodiments, different types of particles may be separated into different channels, e.g., larger particles may be separated from smaller particles, to achieve micro-scale sorting of particles and/or filtering of particles from fluids. Alternatively, in some implementations, the combination of fluid extraction and inertial lift may be used to mix different types of particles. In some cases, both particle separation and shifting between fluids (or particle mixing and shifting between fluids) may be performed together. In another example, the combined fluid extraction and inertial lift forces may be used to focus particles to desired positions within a microfluidic channel. These and other applications may be scaled over large numbers of microfluidic channels to achieve high throughput sorting/filtering of fluids in systems with low device fabrication costs.

In some instances, device 406 can be configured as a particle concentrator capable of causing TEs to transition between fluids. In such embodiments, device 406 includes two inlets and a merging channel for merging the fluids. The merging channel is, in turn, coupled to a particle shifting area that, in some instances, includes different flow regions. During operation, a first fluid containing TEs is introduced through a first inlet and a second fluid containing no particles is introduced through a second inlet. Assuming the fluids are introduced at flow rates corresponding to low Reynolds numbers (and thus laminar flow), there is little mixing between the two different fluids in the merge region of chamber, i.e., the two fluids essentially continue flowing as layers adjacent to one another. As the two fluids enter the channel, particles within the first fluid experience inertial lift forces from structures that are transverse to the direction of flow and that keep the particles within the first channel. An example of a particle concentrator is described in U.S. Pat. No. 10,150,116, which is incorporated herein by reference in its entirety.

At the same time, the increasing width of a second microfluidic channel (due to the slanted channel wall) decreases the fluidic resistance, such that portions of the first fluid (which is nearest to the island structures) are extracted into the second channel at each gap between the structures. Because the first fluid flows as a layer above the second fluid, little to none of the second fluid is extracted into the second channel. After propagating for a sufficient distance past the structures, most of the first fluid is extracted into the second channel, whereas TEs and most or all of the second fluid remain in the first channel. Accordingly, the device 406 can also useful for transferring particles from one fluid to a second different fluid. If the amount of the first fluid flowing through the first inlet is substantially the same as the amount of the second fluid flowing through the second inlet, then the concentration of TEs in the second fluid within the sorting channel (and after extraction of the first fluid) can be kept substantially the same as the concentration of TEs in the first fluid within the first inlet. In some implementations, the propagation distance is long enough so that the second fluid also is extracted into the second microfluidic channel. In that case, the concentration of the TEs in the second fluid within the channel can be increased to a level that is higher than the particle concentration within channel.

As discussed above, in one example, TEs are COCs captured from a FF sample. In this example, denuded oocytes are transferred from a hyaluronidase solution and concentrated into a reduced product volume (e.g., between 50-200 μL). A solution exchange is utilized in device 406 since the denudation process requires the treatment of COCs with hyaluronidase. Despite being a clinical step, prolonged exposure of hyaluronidase to oocytes can sometimes lead to adverse fertility outcomes. After denudation, the enzyme solution around the oocytes is replaced with an exchange solution (e.g. media, cryopreservation solution) using a size-based sorting filter (size cut-off approximately 50 μm). This filter enables the hyaluronidase solution to pass through a discharge outlet, while COCs travels laterally and enter an exchange solution. This stage also enables concentration of COCs in a smaller volume by adjusting the input flowrate of the exchange solution, and the microfluidic resistances of the discharge and product outlets.

### EXAMPLES

### A. COC Capture Device

Preliminary capture experiments (N=4) were conducted with the microfluidic device 110. In these experiments, COCs (N=15-25) were spiked into 5-15 mL of discarded oocyte-free FF fluid. The flowrate through microfluidic devices was set between 0.5-1 mL/min and adjusted by the height of hydrostatic pressure of a vertically standing syringe holding the FF fluid (P=0.35 psi). After the FF fluid was processed and COCs were captured, phosphate buffered saline (PBS) was added to the syringe and an input air pressure between 2-4 psi was applied to release COCs from the microfluidic devices.

Results of the capture experiments are shown in table 700 of FIG. 7. COC capture efficiency of the microfluidic device 110 was measured to be approximately 68%-100%. COC release efficiency of the microfluidic device 110 was measured to be approximately 60%-100% with increased input pressure (pressure purge) and without hyaluronidase treatment. To increase COC release efficiency (or eliminate potential adverse effects of increased flowrate on COCs, input to module 110A (first step) can also be replaced with hyaluronidase. As discussed herein, hyaluronidase weakens binding between secondary entities (CCs) and target entities (oocytes, COCs) and reduce effective COC size due to sacrificial loss of secondary entities. COCs can then be released based on size-based structures in channels 300A and 300B (shown in FIG. 3A) by applying a flow rate that is equal, higher, or lower than the COC capture flow rate.

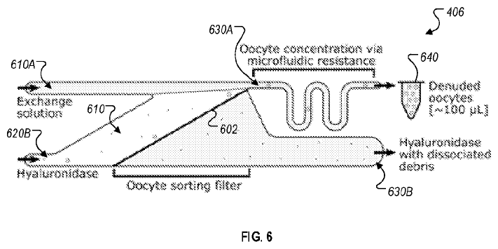

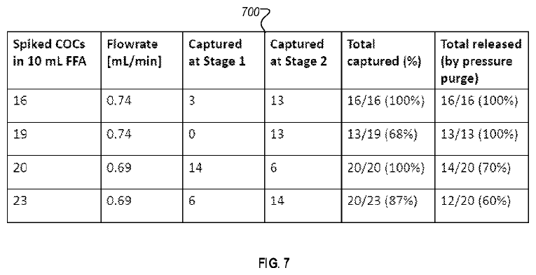

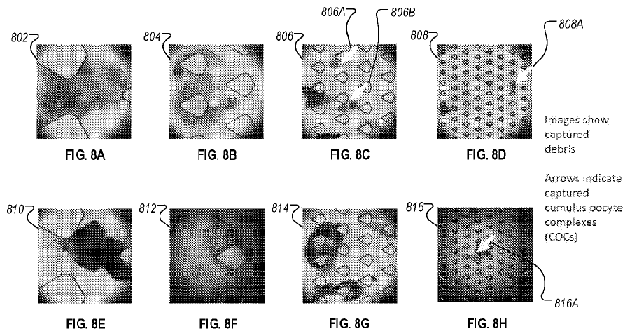

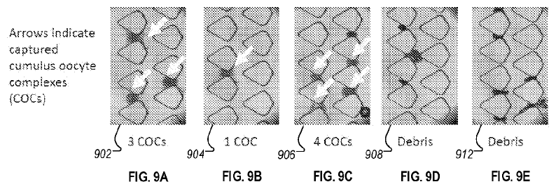

FIGS. 8A to 8H includes microscope images of channel 200 (shown in FIGS. 2A, 2B) collected during capture experiments using the microfluidic device 110. Images 802 and 810 show capture of large tissue debris by structures 202A. Images 804 and 812 show capture of large tissue debris by structures 204A. Images 806 and 814 show capture of large tissue debris and COCs by structures 306A. Images 808 and 816 show capture of capture of large tissue debris and COCs by structures 206A. Arrows 806A, 806B, 808A, and 816A indicate COCs captured in channel 200.

FIGS. 9A to 9E include microscope images of channel 300B (shown in FIG. 3A, 3C) collected during capture experiments using the microfluidic device 110. Image 902 shows the capture of three COCs by structures 312A. Image 904 shows capture three COC by structures 314A. Image 906 shows the capture three COCs by structures 316A. Image 908 shows capture three COCs by structures 318A. Image 912 shows capture of small tissue debris by structures 322A.

### OTHER EMBODIMENTS

Several embodiments of the disclosure have been described. Nevertheless, it will be understood that various modifications may be made without departing from the spirit and scope of the disclosure. Accordingly, other embodiments are within the scope of the following claims.

1. A method of extracting target entities from a sample fluid comprising target entities bound to one or more secondary entities by a specific binding interaction to form target entity-secondary entity complexes that include at least one target entity, the method comprising:
(a) capturing one or more target entity-secondary entity complexes, if any, in the sample fluid using:
(i) a size-based capture mechanism in a microfluidic device comprising a channel having an inlet and an outlet and two or more arrays of structures arranged within the channel between the inlet and the outlet, wherein a first array is arranged in the channel closer to the inlet than a second array, wherein structures in the first array are arranged further apart than structures in the second array, and wherein structures in the second array are arranged at a distance apart that enables capture of target entity-secondary entity complexes, but provides sufficient space to allow target entities and secondary entities to flow through and out of the size-based capture mechanism;
(ii) a binding interaction between the secondary entities and a binding agent in a microfluidic device comprising a channel having an internal surface, an inlet, and an outlet, wherein the binding agent is attached to the internal surface and binds specifically or non-specifically to the secondary entities; or
(iii) both (i) and (ii);
(b) flowing a reagent through the microfluidic device to weaken or break the specific binding interaction between the target entities and the secondary entities or weaken or break binding interactions within or between the secondary entities, thereby releasing the target entities from the target entity-secondary entity complexes; and
(c) capturing a portion of the reagent that includes the target entities.

2. The method of claim 1, wherein:
the sample fluid comprises a biological fluid;
the target entities comprise target cells;
the secondary entities specifically bind to the target cells; and
the reagent comprises a substance that inhibits the specific binding between the target cells and the secondary entities or inhibits binding interactions within or between the secondary entities.

3. The method of claim 1, wherein:
the sample fluid comprises follicular fluid;
the target entities comprise oocytes or cumulus oocyte complexes (COC);
the secondary entities comprise cumulus cells or granulosa cells; and
the reagent comprises hyaluronidase.

4-5. (canceled)

6. The method of claim 1, wherein:
the binding agent binds the secondary entities to the internal surface; and
the reagent specifically weakens specific interactions of the target entities with the secondary entities, thereby releasing the target entities from the target entity-secondary entity complexes.

7-8. (canceled)

9. The method of claim 6, wherein:
the binding agent comprises poly-L-lysine, laminin, follicle-stimulating hormone receptor (FSHR) antibody, luteinizing hormone choriogonadotropin receptor (LHCGR) antibody, or Anti-Mullerian hormone receptor type 2 (AMHR2) antibody; and
the reagent comprises hyaluronidase.

10-11. (canceled)

12. The method of claim 1, wherein:
the channel comprises magnetic beads functionalized for binding to the target entities; and
the method further comprises applying a magnetic field to the microfluidic device to sort magnetic beads bound to the target entities within the channel; and
the portion of the reagent that includes the target entities is captured based on the sorting of magnetic beads bounds to the target entities within the channel.

13-29. (canceled)

30. A microfluidic device for extracting target entities from a sample fluid comprising target entities bound to one or more secondary entities by a specific binding interaction to form target entity-secondary entity complexes that include at least one target entity, the microfluidic device comprising:
a first channel having an inlet, an outlet, and two or more arrays of microposts arranged within the channel between the inlet and the outlet, wherein:
a first micropost array is arranged in the channel closer to the inlet than a second and subsequent micropost arrays,
the second micropost array is arranged in the channel closer to the outlet than the first micropost array,
a subsequent micropost array, if any, is arranged in the channel between the second micropost array and the outlet,
microposts in the subsequent array are arranged (i) closer together than microposts in the second micropost array and (ii) microposts in the second array are closer together than microposts in the first array; and
structures in the second and subsequent micropost arrays are arranged at a distance apart that enables capture of target entity-secondary entity complexes, but provides sufficient space to allow target entities and secondary entities to flow through and out of the five micropost arrays;
a second channel in fluid communication with the first channel and having an inlet, a first outlet, a second outlet, a third outlet, and two rows of filtering structures arranged within the second channel between the inlet and the third outlet, wherein:
a first row of filtering structures is spaced apart in the second channel from a second row of filtering structures such that spacing between the first row and the second row defines a central portion of the second channel,
structures in the first row and the second row are spaced apart along a direction of flow in the central portion so as to permit non-target entities to flow through spaces between the structures and out the first outlet or the second outlet of the second channel, and
the first row and the second row are spaced art perpendicular to direction of flow in the central portion,
the central portion provides sufficient space to allow target entities and secondary entities to flow through and out of the third outlet of the second channel; and
a third channel in fluid communication with the second channel and having an inlet, a product outlet, and three or more micropost arrays of structures arranged within the third channel between the inlet and the product outlet, wherein structures in the three or more micropost arrays are arranged at a distance apart to release target entity-secondary entity complexes through and out of the product outlet of the third channel.

31. (canceled)

32. The microfluidic device of claim 30, wherein:
structures included in the first row and the second row of the second channel are sized to have a length between 650-850 μm along a direction of flow in the central portion;
structures included in the first row and the second row of the second channel are spaced apart between 30-60 along a direction of flow of in the central portion; and
the central portion has a length between 400-600 μm perpendicular to a direction of flow in the central portion.

33-34. (canceled)

35. The microfluidic device of claim 30, wherein:
the three or more micropost arrays of structures arranged within the third channel comprises five micropost arrays of structures;
a first array of the five micropost arrays of structures is arranged in the third channel closer to the inlet than a second array of the five micropost arrays of structures;
a third array of the five micropost arrays of structures is arranged in the third channel between the first array and the second array;
a fourth array of the five micropost arrays of structures is arranged in the third channel between the third array and the second array;
a fifth array of the five micropost arrays of structures is arranged in the third channel between the fourth array and the second array;
structures included in the first array are arranged further apart than structures in the second array;
structures included in the third array are separated by gap sizes that are (i) larger than gap sizes between the structures included in the second array, the fourth array, and the fifth array, and (ii) smaller than gap sizes between the structures included in the first array; and
structures included in the fourth array are separated by gap sizes that are (i) larger than gap sizes between the structures included in the second array and the fifth array, and (ii) smaller than gap sizes between the structures included in the third array.
structures included in the fifth array are separated by gap sizes that are (i) larger than gap sizes between the structures included in the second array, and (ii) smaller than gap sizes between the structures included in the fourth array.

36. The microfluidic device of claim 35, wherein:
the structures included in the first array have gap sizes between structures of 95-145 μm;
the structures included in the second array have gap sizes between structures of 5-55 μm;
the structures included in the third array have gap sizes between structures of 75-125 μm;
the structures included in the fourth array have gap sizes between structures of 55-105 μm; and
the structures included in the fifth array have gap sizes between structures of 25-75 μm.

37. The microfluidic device of claim 36, wherein a height of the structures included in the first array, the second array, the third array, the fourth array, and the fifth array is between 100-1000 μm.

38. (canceled)

39. A method of extracting oocytes from a sample fluid comprising cumulus oocyte complexes (COCs), the method comprising:
(a) capturing one or more COCs, if any, in the sample fluid using a microfluidic device comprising:
(i) a size-based capture mechanism in the microfluidic device, wherein the microfluidic device comprises a channel having an inlet, an outlet, and two or more arrays of structures arranged within the channel between the inlet and the outlet, wherein a first array is arranged in the channel closer to the inlet than a second array, wherein structures in the first array are arranged further apart than structures in the second array, and wherein structures in the second array are configured and arranged at a distance apart that enables capture of COCs, but provides sufficient space to allow cells and/or debris smaller than COCs to flow through and out of the size-based capture mechanism; or
(ii) a binding agent that provides a binding interaction with cumulus cells and/or granulosa cells in the COCs within the microfluidic device, wherein the microfluidic device comprises a channel having an internal surface, an inlet, and an outlet, wherein the binding agent is attached to the internal surface and binds specifically or non-specifically to the cumulus cells and/or granulosa cells; or
(iii) both (i) and (ii);
(b) flowing a reagent solution through the microfluidic device to weaken or break a specific binding interaction between the oocytes and the cumulus cells and/or between the oocytes and the granulosa cells, thereby releasing the oocytes from the COCs; and
(c) capturing a portion of the reagent that includes the oocytes.

40. (canceled)

41. The method of claim 39, wherein the binding agent comprises poly-L-lysine and/or laminin.

42. The method of claim 39, wherein the binding agent comprises a specific binding agent that specifically binds to cumulus cells and/or granulosa cells.

43. The method of claim 42, wherein the specific binding agent comprises an antibody that specifically binds to cumulus cells and/or granulosa cells.

44. The method of claim 43, wherein the antibody comprises a follicle-stimulating hormone receptor (FSHR) antibody, luteinizing hormone choriogonadotropin receptor (LHCGR) antibody, or Anti-Mullerian hormone receptor type 2 (AMHR2) antibody.

45. The method of claim 39, wherein the reagent solution comprises hyaluronidase.

46. The method of claim 39, further comprising, before step (a), filtering and removing from the sample fluid at least some cells and/or debris smaller than about 50 microns.

47. The method of claim 39, further comprising, after step (b) and before or after step (c), denuding the oocytes from any remaining surrounding cumulus cells and/or granulosa cells.

48. The method of claim 39, further comprising, after step (c) or after a denuding step, transferring the oocytes from the reagent solution and concentrating the oocytes into another, different solution.
